(19) United States
(12) Patent Application Publication
(10) Pub. No.: US 2026/0162270 A1
(43) Pub. Date: Jun. 11, 2026

(54) MEDICAL IMAGE SEGMENTATION METHOD AND APPARATUS PERFORMING THE SAME

(71) Applicant: ONCOSOFT INC., Seoul (KR)

(72) Inventors: Jin Sung KIM, Seoul (KR); Seong Gong MOON, Seoul (KR); Jae Hee CHUN, Seoul (KR)

(73) Assignee: ONCOSOFT INC., Seoul (KR)

(21) Appl. No.: 19/110,428

(22) PCT Filed: Apr. 18, 2024
(86) PCT No.: PCT/KR2024/005218

§ 371 Date: Mar. 11, 2025

(30) Foreign Application Priority Data
Aug. 29, 2023 (KR) 10-2023-0113581

(51) Int. Cl. G06T 7/11

(52) U.S. Cl. CPC G06T 7/11; G06T 2207/10081; G06T 2207/20081

(57) ABSTRACT

The present disclosure is a method performed by a processor of a medical image segmentation apparatus including the steps of: acquiring a medical image of a subject; determining a first region corresponding to a target area by inputting the medical image to a first prediction model trained to predict a target area with a medical image as an input; generating a plurality of sub volume data using the first region; determining a second region corresponding to a target area in the first region from the plurality of sub volume data by inputting the plurality of sub volume data to a prediction model trained to predict any one target area with a 3D medical image as an input; and a step of providing a second region corresponding to the target area in the medical image.

### TECHNICAL FIELD

The present disclosure relates to a medical image segmentation method and an apparatus performing the same.

### BACKGROUND ART

Generally, in order to determine whether a subject has a disease in a target area, medical images obtained by capturing the target area of the subject using medical imaging tests (for example, X-ray, ultrasound, computed tomography (CT), angiography, positron emission tomography (PET-CT), single photon emission computed tomography (SPECT-CT), or magnetic resonance imaging (MRI) test) are used. Medical staff has been performing diagnosis by visually examining medical images to identify a target area and determining whether there is a disease in the target area (for example, presence of a tumor).

However, noises may be generated in the medical image itself due to the performance of the imaging device and the movement of the subject. If the target area (for example, an organ or a tumor existing in the organ) is identified only based on the opinion of the medical staff, even for the same medical image, there may be differences in opinions depending on the skills or experiences of the medical staff.

Accordingly, methods for predicting a target area from the medical images using an artificial neural network model trained to predict the target area based on the medical images are being widely used.

However, the artificial neural network model is configured to predict an area where a target area, that is actually 3D, exists using 2D slice-unit medical images. Accordingly, there is a problem that the accuracy and the reliability of the prediction result are low in some slices of the artificial neural network model based on 2D slices.

The background of the present disclosure is described for easier understanding of the present disclosure. It should not be understood to admit the matters described in the background of the present disclosure as a prior art.

### DISCLOSURE

### Technical Problem

In the related art, a model for segmenting only a target area using a 3D medical image has been disclosed, but the unit of 3D learning data is so small that learnable information is limited.

Further, if various types of target areas are to be learned, an amount of data required for the learning is too much, which becomes a limitation in the learning.

Accordingly, a method is demanded to minimize a computational amount of the artificial neural network model while improving the accuracy and the reliability of the prediction results to resolve the uncertainty of the artificial neural network model.

As a result, inventors of the present disclosure configure a prediction model which quickly recognizes the presence of a target area and a type of the target area and accurately recognizes a specific position of each of the plurality of slices which configures a medical image and a medical image segmentation method using the same.

Objects of the present disclosure are not limited to the above-mentioned objects, and other objects, which are not mentioned above, can be clearly understood by those skilled in the art from the following descriptions.

### Technical Solution

In order to achieve the objects as described above, a medical image segmentation method according to an exemplary embodiment of the present disclosure is provided. The method is a method performed by a processor of a medical image segmentation apparatus including acquiring a medical image of a subject; determining a first region corresponding to a target area by inputting the medical image to a first prediction model trained to predict a target area with a medical image as an input; generating a plurality of sub volume data using the first region; determining a second region corresponding to a target area in the first region from the plurality of sub volume data by inputting the plurality of sub volume data to a prediction model trained to predict any one target area with a 3D medical image as an input; and providing a second region corresponding to the target area in the medical image.

According to a feature of the present disclosure, the step of determining a second region may be a step of determining a plurality of second regions corresponding to two or more different target areas according to a type of a target area corresponding to the first region.

According to another feature of the present disclosure, the sub volume data may include data regarding a moving direction of voxels configuring the sub volume data with respect to any one axis.

According to still another feature of the present disclosure, the step of generating sub volume data may be a step of generating a plurality of sub volume data by laminating a plurality of slices configuring the medical image at a predetermined height and segmenting the slices in a direction perpendicular to a plane of the slice.

According to still another feature of the present disclosure, prior to generating the sub volume data, the method may further include a step of inputting the medical image to a classification model trained to classify a type of the target area with the medical image as an input; a step of determining a type of the target area included in each of the plurality of slices configuring the medical image; and a step of grouping the plurality of slices for every target area, according to a classification result.

According to still another feature of the present disclosure, the step of generating a region corresponding to the target area may be a step of providing a user interface screen including a segmentation model selection area to select a target area to be segmented in the medical image and a segmentation result display area to display a region corresponding to the target area.

According to still another feature of the present disclosure, the step of providing a region corresponding to the target area may further include: a step of combining volume data having a probability value corresponding to the target area equal to or higher than a predetermined value from each of the plurality of sub volume data to display the volume data on the user interface screen, using the prediction model.

According to still another feature of the present disclosure, the step of providing a region corresponding to the target area may further include: a step of displaying each of the plurality of sub volume data on the user interface screen with a different transparency according to a probability value corresponding to a target area, using the prediction model.

According to still another feature of the present disclosure, in the step of generating the sub volume data, the plurality of sub volume data is generated with respect to an axis including any one plane, among axial, coronal, and sagittal planes and the step of providing a region corresponding to the target area may further include: a step of acquiring any one reference plane to display the target area, among the axial, coronal, and sagittal planes, through the user interface screen; and a step of rendering a region corresponding to the target area based on the reference plane.

According to still another feature of the present disclosure, prior to the step of acquiring a medical image, the method may further include a step of acquiring learning data having a different slice thickness according to a type of the medical image; a step of generating a learning dataset by bootstrapping a different number of times for every thickness of the learning data; and a step of generating the prediction model configured to predict any one target area based on the learning dataset.

In order to achieve the objects as described above, a medical image segmentation apparatus according to another exemplary embodiment of the present disclosure is provided. The apparatus is configured to acquire a medical image of a subject, determine a first region corresponding to a target area by inputting the medical image to a first prediction model trained to predict a target area with a medical image as an input, generate a plurality of sub volume data using the first region, determine a second region corresponding to a target area in the first region from the plurality of sub volume data by inputting the plurality of sub volume data to a prediction model trained to predict any one target area with a 3D medical image as an input, and provide a second region corresponding to the target area in the medical image.

Other detailed matters of the exemplary embodiments are included in the detailed description and the drawings.

### Advantageous Effects

According to the present disclosure, the medical image segmentation apparatus expands learnable information areas by using a plurality of prediction models for predicting a target area, rather than using one prediction model that inputs a cubic 3D image. Further, the medical image segmentation apparatus increase a learning efficiency by predicting a target area with only a minimum number of learning data.

According to the present disclosure, the medical image segmentation apparatus improves the prediction accuracy of the model by not only inputting only 2D images configuring the medical image to a prediction model predicting a target area, but also inputting front and rear image information of sequentially captured 2D images. Specifically, according to the present disclosure, the medical image segmentation apparatus provides data about a change (for example, a direction of movement for any one specified pixel) of a pixel with respect to a plurality of pixels configuring a 2D image configuring a medical image, thereby improving the prediction accuracy of a model for the entire area of the 2D image.

According to the present disclosure, before the medical image segmentation apparatus predicts the target area, the medical images are linearly classified using a trained classification model, thereby providing a meaningful result where a prediction result using the prediction model is not biased in one direction.

According to the present disclosure, the medical image segmentation apparatus accurately predicts a position of the target area in the medical image and visually displays it to help medical staff to diagnose the target area.

The effects according to the present disclosure are not limited to the contents exemplified above, and more various effects are included in the present disclosure.

### DESCRIPTION OF DRAWINGS

FIG. 1 is a block diagram illustrating a configuration of a medical image segmentation system according to an exemplary embodiment of the present disclosure.

FIG. 2 is a block diagram illustrating a configuration of a medical staff device according to an exemplary embodiment of the present disclosure.

FIG. 3 is a block diagram illustrating a configuration of a medical image segmentation apparatus according to an exemplary embodiment of the present disclosure.

FIG. 4 is a schematic flowchart illustrating a medical image segmentation method according to an exemplary embodiment of the present disclosure.

FIG. 5 is a schematic view for explaining a medical image segmentation method according to an exemplary embodiment of the present disclosure.

FIGS. 6A and 6B are exemplary views of a user interface screen which displays a medical image segmentation result according to an exemplary embodiment of the present disclosure.

FIG. 7 is an exemplary view of a user interface screen which uses a prediction model for medical image segmentation according to an exemplary embodiment of the present disclosure.

FIG. 8 is a schematic view for explaining a method for training a prediction model according to an exemplary embodiment of the present disclosure.

FIG. 9 is a schematic view for explaining an operation method of a prediction model which segments a medical image according to an exemplary embodiment of the present disclosure.

### BEST MODE

Advantages and characteristics of the present disclosure and a method of achieving the advantages and characteristics will be clear by referring to preferable exemplary embodiments described below in detail together with the accompanying drawings. However, the present disclosure is not limited to the following exemplary embodiments but may be implemented in various different forms. The exemplary embodiments are provided only to complete the disclosure of the present disclosure and to fully provide a person having ordinary skill in the art to which the present disclosure pertains with the category of the disclosure. In the description of drawings, like reference numerals denote like components.

In this specification, the terms “have”, “may have”, “include”, or “may include” represent the presence of the characteristic (for example, a numerical value, a function, an operation, or a component such as a part”), but do not exclude the presence of additional characteristic.

In this specification, the terms “A or B”, “at least one of A or/and B”, or “at least one or more of A or/and B” may include all possible combinations of enumerated items. For example, the terms “A or B”, “at least one of A and B”, or “at least one of A or B” may refer to an example which includes (1) at least one A, (2) at least one B, or (3) all at least one A and at least one B.

Although the terms “first”, “second”, and the like, may be used herein to describe various components regardless of an order and/or importance, the components are not limited by these terms. These terms are only used to distinguish one component from another. For example, a first user device and a second user device may refer to different user devices regardless of the order or the importance. For example, without departing from the scope of the present disclosure, a first component may be referred to as a second component, and similarly, a second component may be referred to as a first component.

When a component (for example, a first component) is referred to as being “operatively or communicatively coupled with/to” or “connected to” another component (for example, a second component), it can be understood that the component is directly connected to the other element, or connected to the other element via another component (for example, a third component). In contrast, when a component (for example, a first component) is referred to as being “directly coupled with/to” or “directly connected to” another component (for example, a second component), it is understood that there may be another component (for example, a third component) between the components.

The terms “configured to (or set to)” may be exchangeably used with “suitable for”, “having the capacity to”, “designed to”, “adapted to”, “made to”, or “capable of” depending on the situation. The terms “configured to (or set to)” may not necessarily mean only “specifically designed to” in a hardware manner. Instead, in some situations, the terms “a device configured to” may mean that the device “is capable of” something together with another device or components”. For example, the terms “a processor configured (or set) to perform A, B, and C” may refer to a dedicated processor (for example, an embedded processor) configured to perform the corresponding operation or a generic-purpose processor (for example, a CPU or an application processor) which is capable of performing the operations by executing one or more software programs stored in a memory device.

The terms used in this specification are merely used to describe a specific exemplary embodiment, but do not intend to limit the scope of another exemplary embodiment. A singular form may include a plural form if there is no clearly opposite meaning in the context. Terms used herein including technical or scientific terms may have the same meaning as commonly understood by those skilled in the art. Among the terms used in this specification, terms defined in the general dictionary may be interpreted as having the same or similar meaning as the meaning in the context of the related art, but are not ideally or excessively interpreted to have formal meanings unless clearly defined in this specification. In some cases, even though the terms are defined in this specification, the terms are not interpreted to exclude the exemplary embodiments of the present specification.

The features of various exemplary embodiments of the present disclosure can be partially or entirely bonded to or combined with each other and can be interlocked and operated in technically various ways understood by those skilled in the art, and the exemplary embodiments can be carried out independently of or in association with each other.

For clarity of interpretation of the present specification, terms used in the present specification will be defined below.

The term used in the present specification, a “medical image” may be a 2D image configured by a plurality of cuts (or slices) of the subject. Specifically, the medical image in the present specification may be an enhanced and non-enhanced CT images which are generated according to DICOM standards. For example, the medical image may include a head and neck image including fully from a skull vertex to a lung apex, a chest image including fully from thyroid to a liver dome, an abdomen image including full lumbar vertebrae/L1 spine located 3 cm cranially from the liver dome, and a pelvic image including full ischium located 3 cm cranially from the lumbar vertebrae/Li spine.

The term used in the present specification, “first prediction model” may be a model trained to predict a region corresponding to any one target area with a medical image as an input. Specifically, in the present specification, the first prediction model may be a model trained to predict a first region corresponding to a target area in slices which configure the medical image. For example, the first prediction model may be a model trained to output a region which is predicted that a target area, such as a brain, a neck, a chest, an abdomen, or a pelvis is located as a box shape.

The term used in the present specification, “second prediction model” may be a model trained to predict a region corresponding to any one target area with a 3D medical image as an input. Specifically, the second prediction model in the present specification may be a model trained to predict whether a 3D slab or cube corresponds to the target area. For example, the second prediction model may be a model trained to predict whether each of a plurality of sub volume data is a second region corresponding to a target area, such as a brain, a neck, a chest, an abdomen, or a pelvis. In the meantime, the second region which is predicted by the second prediction model may be included in the first region. The first region may include a plurality of second regions depending on a type of the target area predicted from the first region. As another example, the second prediction model may be a model trained to predict which organ among a plurality of organs which is sequentially disposed in a part of the body of the subject corresponds to each of the plurality of sub volume data. Further, as another example, the second prediction model may be a model trained to predict whether each of the plurality of volume data corresponds to an area which is suspected of having a disease in an organ, a bone, or a muscle. Here, the area which is suspected of having a disease may be an area which is suspected of having a tumor and the medical image may be a medical image obtained by capturing the head and neck, the chest, the abdomen, and the pelvis.

In various exemplary embodiments, the first and second prediction models may be implemented by at least one model of a fully convolutional network (FCN) having convolutional neural network (CNN) based VGG net, R, DenseNet, and an encoder-decoder structure, a deep neural network (DNN) such as SegNet, DeconvNet, DeepLAB, V3+, or U-net, or SqueezeNet, Alexnet, ResNet18, MobileNet-v2, GoogLeNet, Resnet50, Resnet101, and Inception-v3, or may be implemented with two or more ensemble models.

In various exemplary embodiments, the second prediction model may be a model which is trained with learning data having a different slice thickness depending on a type of the medical image. In the present disclosure, a medical image segmentation apparatus performs training by resampling the slice without performing the pre-processing process for increasing or reducing the slice thickness in order to unify the slice thickness according to a reference value. Specifically, the second prediction model uses slap data with a size of 256×256×16 or cubic data with a size of 96×96×96 as sub volume data and bootstraps a different number of times according to a thickness of slice input as learning data so that the second prediction model may be trained without losing the medical image. For example, when a slice with a thickness of 3 mm is input at the beginning of the learning, the second prediction model may perform the training by laminating slices to generate sub volume data in the unit of 48 mm. When a slice with a thickness of 5 mm is input at the beginning of the next learning, the second prediction model may perform the training by laminating slices to generate sub volume data in the unit of 90 mm. Such learning may be performed as many epochs as specified.

The term used in the present specification, “classification model” may be a classification model trained to segment a type of target area with a medical image as an input. Specifically, the classification model may be a model trained to classify a target area of at least one of a brain, a neck, a chest, an abdomen, and a pelvic. The medical image segmentation apparatus may group sequentially captured slices according to each type of target area by using the classification model.

In various exemplary embodiments, prior to predicting whether it is a target area of the volume data using the first and second prediction models, the classification model is used to increase the computation efficiency rather than solely using the first and second prediction models.

Hereinafter, the present disclosure will be described in detail by explaining preferred exemplary embodiments of the present disclosure with reference to the accompanying drawings.

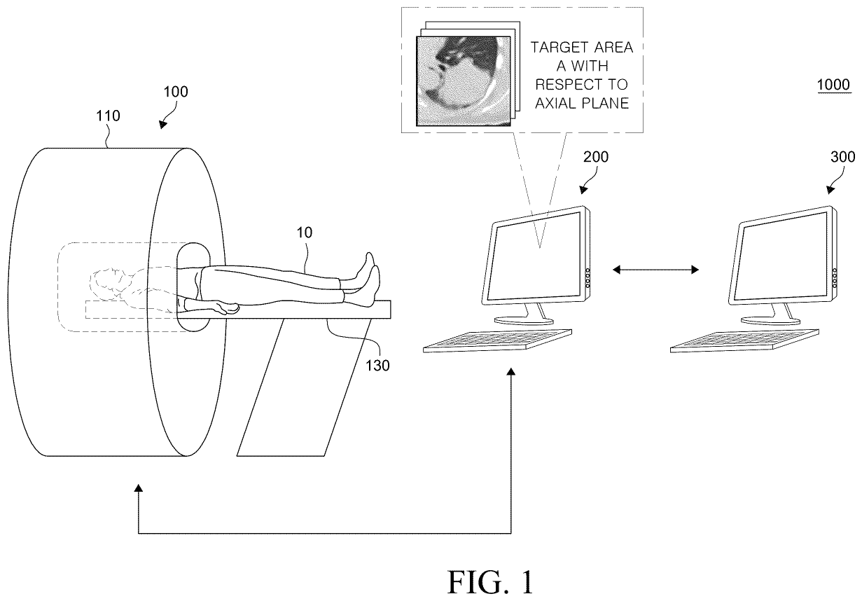

FIG. 1 is a block diagram illustrating a configuration of a medical image segmentation system according to an exemplary embodiment of the present disclosure.

Referring to FIG. 1, the medical image segmentation system 1000 may be a system configured to segment only a target area from the medical image. Here, when the target area is segmented, it means that a region corresponding to the target area is displayed in the medical image. To this end, the medical image segmentation system 1000 may include an imaging device 100 which captures a part of a body of a subject, a medical staff device 200 which identifies a region corresponding to the target area and diagnoses the subject, and a medical image segmentation apparatus 300 which segments a region corresponding to the target area in the medical image.

The imaging device 100 is a device which acquires a medical image for a target area of the subject 10, such as human or animals, and may include a cylindrical bore 110 into which the subject 10 is carried and a transport device 130 in which the subject 10 is seated to be carried inside. Here, the target area may include various organs, bones, and muscles, such as a brain, a neck, a chest, an abdomen, and a pelvis. The imaging device 100 may acquire a medical image for the subject 10 by irradiating X-ray which projects the subject 10, toward the subject 10.

In various exemplary embodiments, the imaging device 100 may acquire a gray scale or RGB 2D image, a still image configured by one cut, and a video configured by a plurality of cuts, as a medical image for predicting a region corresponding to the target area.

The medical staff device 200 may be a device which transmits a segmentation request of the medical image to the medical image segmentation apparatus 300 and displays a medical image segmentation result. For example, the medical staff device 200 may include a smart phone, a tablet personal computer (PC), a notebook, and a PC.

In various exemplary embodiments, the medical staff device 200 may display a medical image segmentation result which is provided by the medical image segmentation apparatus 300 through a user interface screen. For example, the medical image segmentation result may be based on a single plane, and the area corresponding to the target area in each of plurality of slices may be highlighted with a different color.

In various exemplary embodiments, the medical staff device 200 is configured to install or execute a web, a mobile application, or a program provided by the medical image segmentation apparatus 300 to perform a series of the medical image segmentation method performed by the medical image segmentation apparatus 300 to be described below.

The medical image segmentation apparatus 300 may acquire a medical image of the subject from the imaging device 100 and predict a region corresponding to the target area based on the medical image. To this end, the medical image segmentation apparatus 300 may include a general purpose computer, a laptop, and a data server which are capable of performing the deep learning on the medical image and analyzing the medical image.

In various exemplary embodiments, the medical image segmentation apparatus 300 inputs a plurality of slices which configures a medical image to a first prediction model which is trained to predict any one target area with the medical image as an input to determine a first region corresponding to the target area in each of the plurality of slices. For example, the medical image segmentation apparatus 300 may output a first region of each of the slices corresponding to a target area, such as a brain, a neck, a chest, an abdomen, or a pelvis in a box form.

In the present disclosure, the medical image segmentation apparatus 300 may not only predict a region of each of the plurality of slices which configures the medical image corresponding to the target area, but also generate a plurality of sub volume data using the plurality of slices and predict a region corresponding to the target area based on the sub volume data. Here, the sub volume data may be generated by laminating the plurality of slices which configures the medical image at a predetermined height and segmenting the plurality of slices in a direction perpendicular to a plane of the slice. For example, the sub volume data may be slab data with a size of 256×256×16 and as another example, the sub volume data may be cubic data with a size of 96×96×96. Further, voxels which configure the sub volume data may have sizes of 2×2×3 mm³ or 0.7×0.7×1.0 mm³. Further, Voxels may have various sizes within the range of 0.7×0.7×1.0 mm³ to 2×2×3 mm³.

The medical image segmentation apparatus 300 may sequentially utilize two prediction models including a prediction model which has a 2D image as an input and a prediction model which has a 3D image as an input. The medical image segmentation apparatus 300 of the present disclosure may increase a precision of the prediction more than the prediction of the target area obtained by solely using a prediction model with slices as an input and expand a variety of a prediction result more than the prediction obtained by solely using the prediction model with 3D volume data as an input.

In the present disclosure, a plurality of sub volume data may include data regarding a moving direction of voxels which configure the sub volume data with respect to any one axis and increase the accuracy of the prediction result, more than the prediction of the target area only with the slice unit. For example, when the sub volume data is slab data, each sub volume data may include data regarding the moving direction of the voxels in a direction perpendicular to the horizontal plane (axial). As another example, when the sub volume data is cubic data, each sub volume data may include data regarding the moving direction of the voxels in at least one of axial, coronal, and sagittal directions.

In various exemplary embodiments, the medical image segmentation apparatus 300 may determine a second region corresponding to the target area from the plurality of sub volume data, by inputting the plurality of sub volume data to the second prediction model which is trained to predict any one target area with a 3D medical image as an input. In other words, the medical image segmentation apparatus 300 may predict whether each of the plurality of sub volume data corresponds to the target area. For example, the medical image segmentation apparatus 300 may predict whether each of the plurality of sub volume data corresponds to a target area, such as a brain, a neck, a chest, an abdomen, or a pelvis.

To this end, the medical image segmentation apparatus 300 may include a plurality of second prediction models which predicts a specific region of each of a brain, a neck, a chest, an abdomen, and a pelvis. The medical image segmentation apparatus 300 may use any one second prediction model, among the plurality of second prediction models, in accordance with a region predicted by the first prediction model. That is, the medical image segmentation apparatus 300 may schematically determine a first region of a target area using the first prediction model and determine a second region indicating a specific shape of the target area using the second prediction model. Accordingly, the first region and the second region determined by the first prediction model and the second prediction model may correspond to the same target area. However, depending on positions of organs in the body, the first region and the second region may correspond to different target areas and a plurality of second regions may be included in the first region. For example, the first region may be a lung and the plurality of second regions may be a left lung and a right lung.

In various exemplary embodiments, the medical image segmentation apparatus 300 may predict a target area, such as gland thyroid, cavity oral, bone mandible, left submandibular gland, right submandibular gland, pharynx, larynx, left parotid, right parotid, left temporomandibular joint, right temporomandibular joint, left brachial plexus, and right brachial plexus in the neck, through the first and second prediction models. As another example, the medical image segmentation apparatus 300 may predict a target area, such as gland thyroid, left lung, right lung, heart, left humerus, right humerus, trachea, left bronchus, right bronchus, left brachial plexus, and right brachial plexus in the chest, through the first and second prediction models. As still another example, the medical image segmentation apparatus 300 may predict a target area, such as river, pancreas, gallbladder, spleen, left kidney, and right kidney in the abdomen, through the first and second prediction models. As still another example, the medical image segmentation apparatus 300 may predict a target area, such as spinal cord, esophagus, stomach, duodenum, cauda equina, and bowel in a continuous organ, through the first and second prediction models.

In various exemplary embodiments, the medical image segmentation apparatus 300 inputs a medical image of the subject to a classification model which is trained to classify a type of a target area with the medical image as an input to determine a type of the target area included in each of the plurality of slices which configures the medical image. For example, the classification model may determine whether the slice includes any one target area, among a brain, a neck, a chest, a abdomen, a pelvis, an optic, a heart, a breast, and bowels. The medical image segmentation apparatus 300 may group the plurality of slices for every target area, according to the classification result.

In various exemplary embodiments, the medical image segmentation apparatus 300 displays a region corresponding to the target area in the medical image by combining results predicted based on the plurality of sub volume data to provide the result to the medical staff device 200. For example, the medical image segmentation apparatus 300 may provide a user interface screen including a segmentation result display area in which a region corresponding to the target area is displayed, to the medical staff device 200. Here, the user interface screen may include a segmentation model selection area to select a target area to be segmented in the medical image and by doing this, only a specific target area that the medical staff wants to identify from the medical image may be emphasized and displayed. Further, the medical image segmentation apparatus 300 predicts a region corresponding to the target area based on the plurality of sub volume data so that the medical image segmentation apparatus 300 may display and provide a target area captured on any one reference plane, among axial, coronal, and sagittal directions, according to the choice of the medical staff.

Until now, the medical image segmentation system 1000 according to the exemplary embodiment of the present disclosure has been described. According to the present disclosure, the medical image segmentation system 1000 accurately predicts and visually displays a position of the target area in the medical image to help medical staff to diagnose the target area.

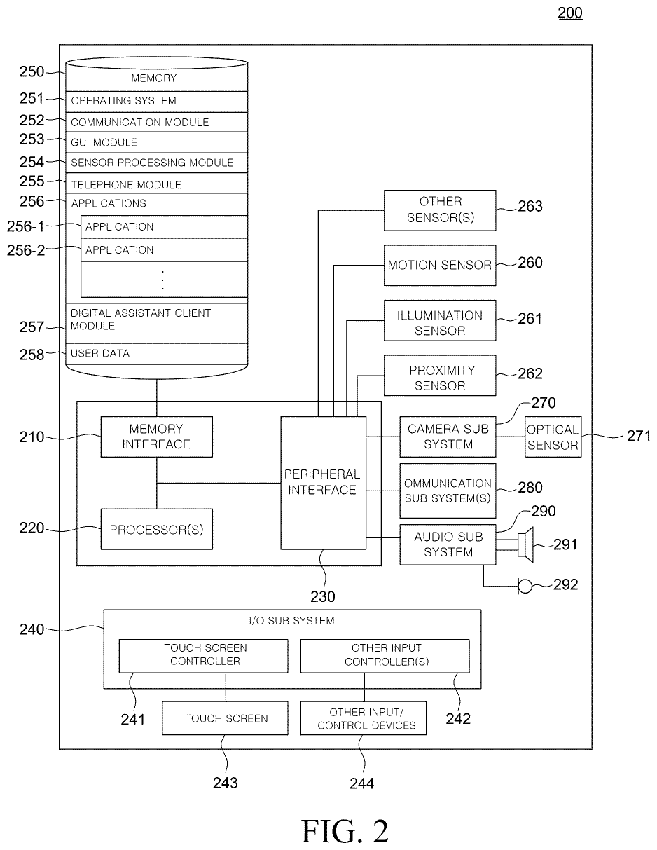

Hereinafter, the medical staff device 200 which utilizes the medical image segmentation result will be described with reference to FIG. 2.

FIG. 2 is a block diagram illustrating a configuration of a medical staff device according to an exemplary embodiment of the present disclosure.

Referring to FIG. 2, the medical staff device 200 may include a memory interface 10, one or more processors 220, and a peripheral interface 230. Various components in the medical staff device 200 may be connected by one or more communication buses or signal lines.

The memory interface 210 is connected to a memory 250 to transmit various data to the processor 220. Here, the memory 250 may include at least one type of storing media of a flash memory type, a hard disk type, a multimedia card micro type, a card type memory (for example, an SD or XD memory), a RAM, an SRAM, a ROM, an EEPROM, a PROM, a network storage, a cloud, and a block chain database.

In various exemplary embodiments, the memory 250 may store a web/app application or a program for segmenting only a region corresponding to the target area from the medical image. Further, the memory 250 may store subject's identification information (for example, age, gender, whether to have a disease), a medical image of the subject, and a region corresponding to the target area in the medical image.

In various exemplary embodiments, the memory 250 may store at least one of an operating system 251, a communication module 252, a graphic user interface module (GUI) 253, a sensor processing module 254, a telephone module 255, and an application module 256. Specifically, the operating system 251 may include an instruction for processing a basic system service and an instruction for performing hardware tasks. The communication module 252 may communicate with at least one of one or more other devices, computers, and servers. The graphic user interface module (GUI) 253 may process a graphic user interface. The sensor processing module 254 may process sensor-related functions (for example, to process a received voice input using one or more microphones 292). The telephone module 255 may process telephone-related functions. The application module 256 may perform various functions of the user application, such as electronic messaging, web browsing, media processing, searching, imaging, or other processing functions. Further, the medical staff device 200 may store one or more software applications 256-1 and 256-2 (for example, a medical image segmentation application) related to any one type of service in the memory 250.

In various exemplary embodiments, the memory 250 may store a digital assistant client module 257 (hereinafter, simply referred to as a DA client module) and accordingly, may store instructions for performing a function of the client of the digital assistant and various user data 258 (for example, user-customized vocabulary data, preference data, or other data such as user's electronic address book).

In the meantime, the DA client module 257 may acquire voice input, text input, touch input and/or gesture input of the user by means of various user interfaces (for example, I/O sub system 240) equipped in the medical staff device 200.

Further, the DA client module 257 may output audio-visual or tactile data. For example, the DA client module 257 may output data formed of a combination of at least two or more of voice, sound, a notice, a text message, a menu, a graphic, a video, an animation, and a vibration. Further, the DA client module 257 may communicate with a digital assistant server (not illustrated) using a communication sub system 280.

In various exemplary embodiments, the DA client module 257 may collect additional information about the surrounding environment of the medical staff device 200 from various sensors, sub systems, and peripheral devices to configure a context associated with the user input. For example, the DA client module 257 may infer the intention of the user by providing context information to the digital assistant server together with the user input. Here, the context information which may be accompanied by the user input may include sensor information, such as light, ambient noises, ambient temperature, an image of the surrounding environment, and a video. As another example, the context information may include a physical state (for example, a device alignment, a device position, a device temperature, a power level, a speed, an acceleration, a motion pattern, or a cellular signal intensity) of the medical staff device 200. As another example, the context information may include information related to a software state of the medical staff device 200 (for example, a process which is being executed in the medical staff device 200, installed program, past and present network activities, a background service, an error log, or resource usage).

In various exemplary embodiments, the memory 250 may include added or deleted instructions. Moreover, the medical staff device 200 may also include an additional configuration, in addition to the configuration illustrated in FIG. 2 or exclude some configurations.

The processor 220 may control an overall operation of the medical staff device 200 and perform various instructions to display a region corresponding to the target area in the medical image through a medical staff or implement a user interface to identify a prediction result provided from the first and second prediction models, by driving the application or the program stored in the memory 250.

The processor 220 may correspond to an arithmetic device such as a central processing unit (CPU) or an application processor (AP). Further, the processor 220 may be implemented as an integrated chip (IC) such as a system of chip (SoC) in which various arithmetic devices which perform machine learning, such as a neural processing unit (NPU), are integrated.

In various exemplary embodiments, the processor 220 may display a target area in the medical image or request segmentation of the target area and display a result thereof, by means of a user interface screen, by an application or a program for medical image segmentation provided by the medical image segmentation apparatus 300.

In various exemplary embodiments, the processor 220 may acquire the medical image from the imaging device 100 and generate a plurality of sub volume data using the medical image. In the meantime, the processor 220 may generate sub volume data only for a region of the target area predicted by the first prediction model, without generating sub volume data for the entire area of the medical image. Specifically, the processor inputs a medical image to the first prediction model trained to predict any one target area with the medical image as an input to determine a first region corresponding to target area. Further, the processor 220 inputs sub volume data generated only for the first region to the second prediction model which is trained to predict any one target area with a 3D medical image as an input to determine a second region corresponding to the target area from the plurality of sub volume data and may provide a region corresponding to the target area in the medical image through the user interface screen.

The peripheral interface 230 is connected to various sensors, sub systems, and peripheral devices to provide data to allow the medical staff device 200 to perform various functions. Here, when the medical staff device 200 performs any function, it is understood that the function is performed by the processor 220.

The peripheral interface 230 may receive data from a motion sensor 260, an illumination sensor (an optical sensor) 261, and a proximity sensor 262 and by doing this, the medical staff device 200 may perform alignment, light, and proximity sensing functions. As another example, the peripheral interface 230 may be provided with data from other sensors 263 (a positioning system-GPS receiver, a temperature sensor, or a biometric sensor), and by doing this, the medical staff device 200 may perform functions related to the other sensors 263.

In various exemplary embodiments, the medical staff device 200 may include a camera sub system 270 connected to the peripheral interface 230 and an optical sensor 271 connected thereto and by doing this, the medical staff device 200 may perform various photographing functions such as taking a picture or recording a video clip.

In various exemplary embodiments, the medical staff device 200 may include a communication sub system 280 connected to the peripheral interface 230. The communication sub system 280 is configured by one or more wired/wireless networks and may include various communication ports, a wireless frequency transceiver, and an optical transceiver.

In various exemplary embodiments, the medical staff device 200 includes an audio sub system 290 connected to the peripheral interface 230 and the audio sub system 290 includes one or more speakers 291 and one or more microphones 292 so that the medical staff device 200 may perform voice-operated functions, such as voice recognition, voice duplication, digital recording, and telephone functions.

In various exemplary embodiments, the medical staff device 200 may include an I/O sub system 240 connected to the peripheral interface 230. For example, the I/O sub system 240 may control the touch screen 243 included in the medical staff device 200 by means of a touch screen controller 241.

For example, the touch screen controller 241 may use any one of a plurality of touch sensing techniques such as a capacitive type, a resistive type, an infrared type, a surface acoustic wave technology, or a proximity sensor array to detect contact and movement of the user or stopping of contact and movement. As another example, the I/O sub system 240 may control the other input/control device 244 included in the medical staff device 200 by means of other input controller(s) 242. As an example, other input controller(s) 242 may control one or more buttons, rocker switches, thumb-wheels, infrared ports, USB ports, and pointer devices such as a stylus.

Until now, the medical staff device 200 according to the exemplary embodiment of the present disclosure has been described. According to the present disclosure, a target area, for example, a position and a size of an organ, a bone, or a tumor, to be diagnosed from the medical image, may be accurately identified using the medical staff device 200 and thus the health condition of the subject may be accurately diagnosed.

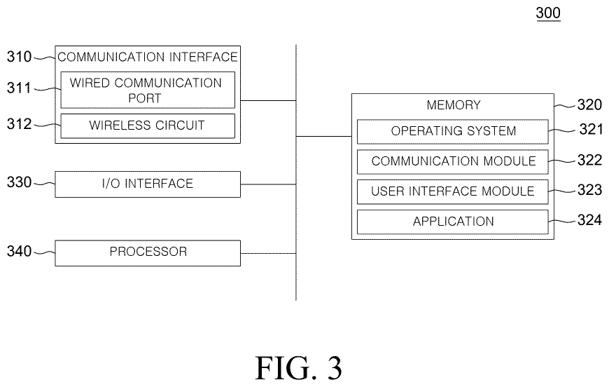

Hereinafter, referring to FIG. 3, a medical image segmentation apparatus 300 which provides a result obtained by segmenting a target image in the medical image will be described with reference to FIG. 3.

FIG. 3 is a block diagram illustrating a configuration of a medical image segmentation apparatus according to an exemplary embodiment of the present disclosure.

Referring to FIG. 3, the medical image segmentation apparatus 300 may include a communication interface 310, a memory 320, an I/O interface 330, and a processor 340 and each configuration may communicate with each other via one or more communication buses or signal lines.

The communication interface 310 is connected to the imaging device 100 and the medical staff device 200 via a wired/wireless communication network to exchange data. For example, the communication interface 310 may receive a medical image of the subject from the imaging device 100 or the medical staff device 200. As another example, the communication interface 310 may transmit a result of predicting a region corresponding to the target area in the medical image to the medical staff device 200 and provide a user interface screen to visually display the result.

In the meantime, the communication interface 310 which enables the transmission/reception of the data includes a wired communication port 311 and a wireless circuit 312 and the wired communication port 311 may include one or more wired interfaces, such as Ethernet, universal serial bus (USB), and fire wire. Further, the wireless circuit 312 may transmit and receive data with external devices by an RF signal or an optical signal. In addition, the wireless communication may use at least one of a plurality of communication standards, protocols, and technologies, such as GSM, EDGE, CDMA, TDMA, Bluetooth, Wi-Fi, VoIP, Wi-Max, or other arbitrary appropriate communication protocols.

The memory 320 may store various data used in the medical image segmentation apparatus 300. For example, the memory 320 may store configurations and learning data of first and second prediction models trained to predict a region corresponding to any one target area with identification information of the imaging device 100 and the medical staff device 200 and a 3D medical image as inputs and a classification model trained to segment a type of target area with a medical image as an input.

In various exemplary embodiments, the memory 320 may include a volatile or nonvolatile recording medium which may store various data, commands, and information. For example, the memory 320 may include at least one type of storage media of a flash memory type, a hard disk type, a multimedia card micro type, a card type memory (for example, an SD or XD memory), a RAM, an SRAM, a ROM, an EEPROM, a PROM, a network storage, a cloud, and a block chain database.

In various exemplary embodiments, the memory 320 may store a configuration of at least one of an operating system 321, a communication module 322, a user interface module 323, and one or more applications 324.

The operating system 321 (for example, an embedded operating system, such as LINUX, UNIX, MAC OS, WINDOWS, VxWorks) may include various software components and drivers which control and manage a general system task (for example, memory management, storage device control, or power management) and support communication between various hardware, firmware, and software components.

The communication module 322 may support communication with other devices through the communication interface 310. The communication module 322 may include various software components for processing data received by a wired communication port 311 or a wireless circuit 312 of the communication interface 310.

The user interface module 323 may receive a request or an input of the user from a keyboard, a touch screen, a mouse, or a microphone via the I/O interface 330 and provide the user interface on the display.

The application 324 may include a program or a module configured to be executed by one or more processors 340. Here, the application for medical image classification and segmentation may be implemented on a server farm.

The I/O interface 330 may connect at least one of input/output devices (not illustrated) of the medical image segmentation apparatus 300, such as a display, a keyboard, a touch screen, and a microphone, to the user interface module 323. The I/O interface 330 may receive the user input (for example, voice input, keyboard input, or touch input) together with the user interface module 323 and process a command in accordance with the received input.

The processor 340 may be connected to the communication interface 310, the memory 320, and the I/O interface 330 to control an overall operation of the medical image segmentation apparatus 300, train the first and second prediction models and the classification model through the application or the program stored in the memory 320, and may perform various commands to segment a target area in the medical image when a new medical image is input.

The processor 340 may correspond to an arithmetic device such as a central processing unit (CPU) or an application processor (AP). Further, the processor 340 may be implemented as an integrated chip (IC) such as a system of chip (SoC) in which various arithmetic devices are integrated. Further, the processor 340 may include a module for calculating an artificial neural network model like a neural processing unit (NPU).

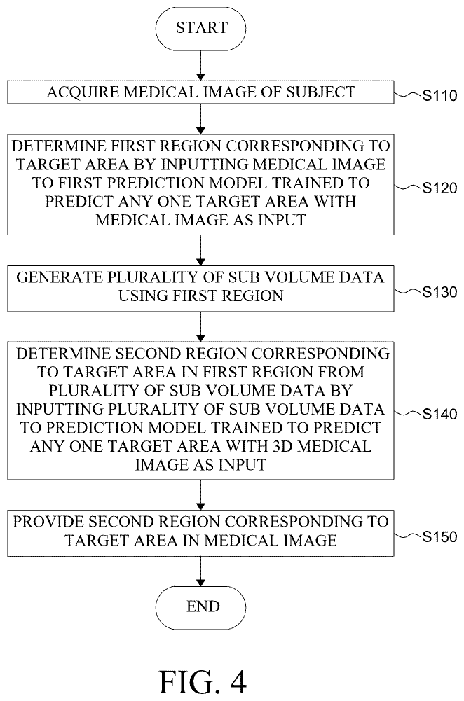

Hereinafter, a method for segmenting a medical image by a processor 340 of the medical image segmentation apparatus 300 will be described with reference to FIGS. 4 to 6.

FIG. 4 is a schematic flowchart illustrating a medical image segmentation method according to an exemplary embodiment of the present disclosure.

Referring to FIG. 4, the processor 340 may acquire a medical image of a subject in step S110. Here, the medical image may be a 2D image configured by a plurality of cuts (slices) which is enhanced and non-enhanced computed tomographic (CT) images. For example, the medical image may include a head and neck image including fully from a skull vertex to a lung apex, a chest image including fully from thyroid to a liver dome, an abdomen image including full lumbar vertebrae/L1 spine located 3 cm cranially from the liver dome, and a pelvic image including full ischium located 3 cm cranially from the lumbar vertebrae/Li spine.

In various exemplary embodiments, the processor 340 divides and groups the plurality of slices which configures the medical image according to a target area to increase a computation efficiency of the second prediction model. Specifically, the processor 340 inputs a medical image of the subject to a classification model which is trained to classify a type of a target area with the medical image as an input to determine the type of the target area included in each of the plurality of slices which configures the medical image. For example, the classification model may determine whether the slice includes any one target area, among a brain, a neck, a chest, an abdomen, and a pelvis. The medical image segmentation apparatus 300 may group the plurality of slices for every target area, according to the classification result.

After the step S110, the processor 340 may input a medical image to the first prediction model trained to predict any one target area with the medical image as an input to determine a first region corresponding to target area in step S120. Specifically, the first prediction model may be a model trained to predict a first region corresponding to a target area in slices which configure the medical image. Accordingly, for example, the processor 340 may output a region which is predicted that a target area, such as a brain, a neck, a chest, an abdomen, or a pelvis is located from each of the slice as a box shape through the first prediction model and determine the region as a first region.

After the step S120, the processor 340 may generate a plurality of sub volume data using the first region in step S130. Specifically, the sub volume data may be generated by laminating the plurality of slices which configures the medical image at a predetermined height and segmenting the plurality of slices in a direction perpendicular to a plane of the slice. For example, the sub volume data may be slab data with a size of 256×256×16 and as another example, the sub volume data may be cubic data with a size of 96×96×96. Further, voxels which configure the sub volume data may have sizes of 2×2×3 mm³ or 0.7×0.7×1.0 mm³. Further, Voxels may have various sizes within the range of 0.7×0.7×1.0 mm³ to 2×2×3 mm³. The processor 340 may generate a plurality of sub volume data with respect to an axis including any one plane, among axial, coronal, and sagittal planes.

In various exemplary embodiments, a plurality of sub volume data may include data regarding a moving direction of voxels which configure the sub volume data with respect to any one axis and increase the accuracy of the prediction result, more than the prediction of the target area in the unit of slices. For example, when the sub volume data is slab data, each sub volume data may include data regarding the moving direction of the voxels in a direction perpendicular to the horizontal plane (axial). As another example, when the sub volume data is cubic data, each sub volume data may include data regarding the moving direction of the voxels in at least one of axial, coronal, and sagittal directions.

After the step S130, the processor 340 may determine a second region corresponding to the target area in the first region from the plurality of sub volume data, by inputting the plurality of sub volume data to the second prediction model which is trained to predict any one target area with a 3D medical image as an input in step S140.

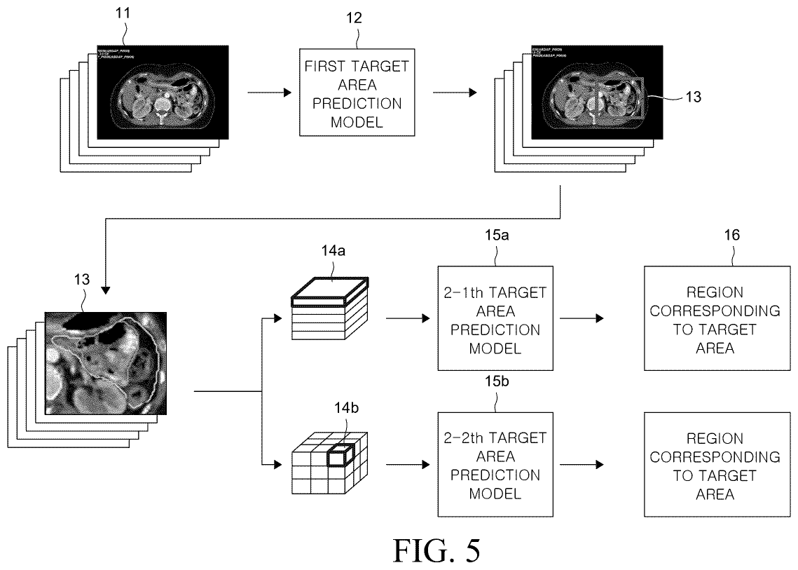

With regard to this, FIG. 5 is a schematic view for explaining a medical image segmentation method according to an exemplary embodiment of the present disclosure.

Referring to FIG. 5, the processor 340 may determine a first region 13 corresponding to the target area by inputting a medical image 11 configured by a plurality of slices to the first prediction model 12. Here, the medical image 11 may be a medical image which is classified to include any one target area, among the head, the neck, the chest, the abdomen, and the pelvis. The processor 340 may generate a plurality of sub volume data by laminating and segmenting only the first region 13 corresponding to the target area. The processor 340 may generate a plurality of sub volume data defined as slab data 14a with a size of 256×256×16 and cubic data 14b with a size of 96×96×96 using the medical image 11.

In various exemplary embodiments, the processor 340 inputs slab data 14a to a 2-1-th target area second prediction model 15a or cubic data 14b to a 2-2-th target area second prediction model 15b depending on a type of generated sub volume data to output a result 16 predicting whether each sub volume data corresponds to the target area. Here, the target area may include an organ, a bone, or a muscle, such as a brain, a neck, a chest, an abdomen, or a pelvis or a tumor which is suspected of being a disease formed around the organ, the bone, or the muscle.

As described above, the processor 340 may schematically determine a first region of a target area using the first prediction model and determine a second region indicating a specific shape of the target area using the second prediction model. Accordingly, the first region and the second region determined by the first prediction model and the second prediction model may correspond to the same target area. However, depending on positions of organs in the body, the first region and the second region may correspond to different target areas and a plurality of second regions may be included in the first region. In other words, the processor 340 may determine a plurality of second regions corresponding to two or more different target areas depending on a type of the target area corresponding to the first region. For example, the first region may be a lung and the plurality of second regions may be a left lung and a right lung.

In the meantime, in order to obtain such a prediction result, the second prediction model may be trained with learning data having a different slice thickness according to a type of the medical image. Specifically, the second prediction model uses slap data with a size of 256×256×16 or cubic data with a size of 96×96×96 as sub volume data so that the processor 340 bootstraps a different number of times according to a thickness of slice input as learning data so that the second prediction model may be trained without losing the medical image. For example, when a slice with a thickness of 3 mm is input at the beginning of the learning, the processor 340 may train the second prediction model by laminating slices to generate sub volume data in the unit of 48 mm and when a slice with a thickness of 5 mm is input at the beginning of the next learning, the processor 340 may train the second prediction model by laminating slices to generate sub volume data in the unit of 90 mm. The processor 340 may perform such learning as many epochs as specified in the memory 320.

In addition, the processor 340 preprocesses a plurality of slices to be input to the second prediction model, that is, slices including only the first region, to be used as learning data. For example, the processor 340 copies one slice to laminate the slices in accordance with a reference thickness of the learning data or linearly interpolates to resample the thickness of the slice in accordance with the reference thickness.

Referring to FIG. 4 again, the processor 340 may provide a second region corresponding to the target area in the medical image in step S150. Specifically, the processor 340 may determine whether the plurality of sub volume data corresponds to each target area using the second prediction model and segment each of the plurality of sub volume data according to the result. The processor 340 may display the second region corresponding to the target area from the plurality of slices which configures the medical image by combining the segmented results.

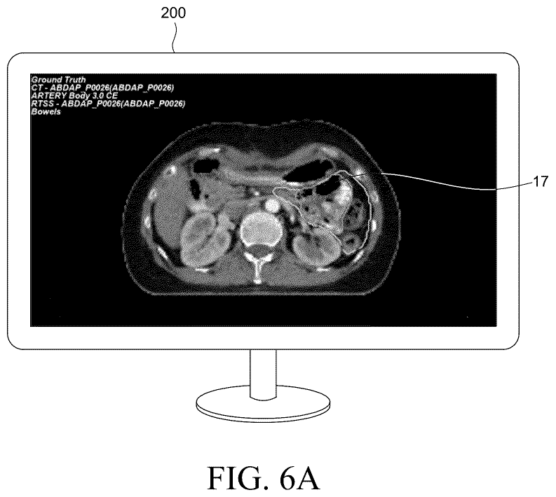

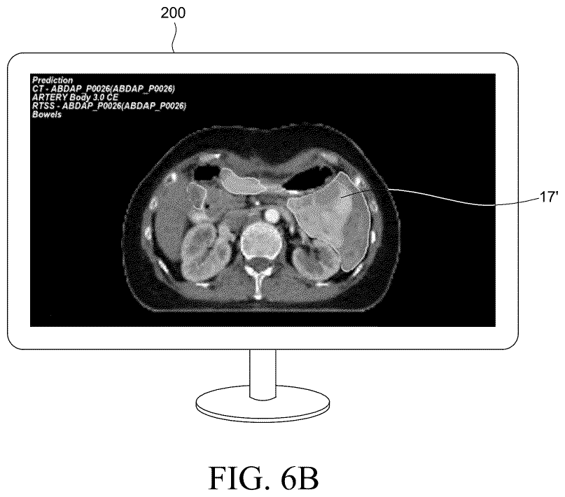

With regard to this, FIGS. 6A and 6B are exemplary views of a user interface screen which displays a medical image segmentation result according to an exemplary embodiment of the present disclosure.

Referring to FIGS. 6A and 6B, the processor 340 displays the second region corresponding to the target area in the medical image to provide it to the medical staff device 200. Specifically, the processor 340 combines volume data having a probability value corresponding to the target area which is equal to or higher than a predetermined value, from each of the plurality of sub volume data, using the second prediction model to display the volume data on the user interface screen. For example, as illustrated in FIG. 6A, the processor 340 may emphasize and display only an edge area 17 of the target area having a probability value corresponding to the target area which is equal to or higher than a predetermined value. Further, the processor 340 may display a region of the medical image which is predicted as a target area with different colors depending on the type.

Further, the processor 340 may display each of the plurality of sub volume data on the user interface screen with a different transparency according to the probability value corresponding to the target area, using the second prediction model. For example, as illustrated in FIG. 6B, the processor 340 may emphasize and display a region 17′ which is predicted as a target area in the medical image with different transparencies according to the probability value. The processor 340 may control transparency to be lowered as probability value increases so that the target area to be highlighted in the medical image.

In various exemplary embodiments, the processor 340 may provide the user interface screen including a segmentation model selection area to select a target area to be segmented in the medical image and a segmentation result display area 15 to display a region corresponding to the target area.

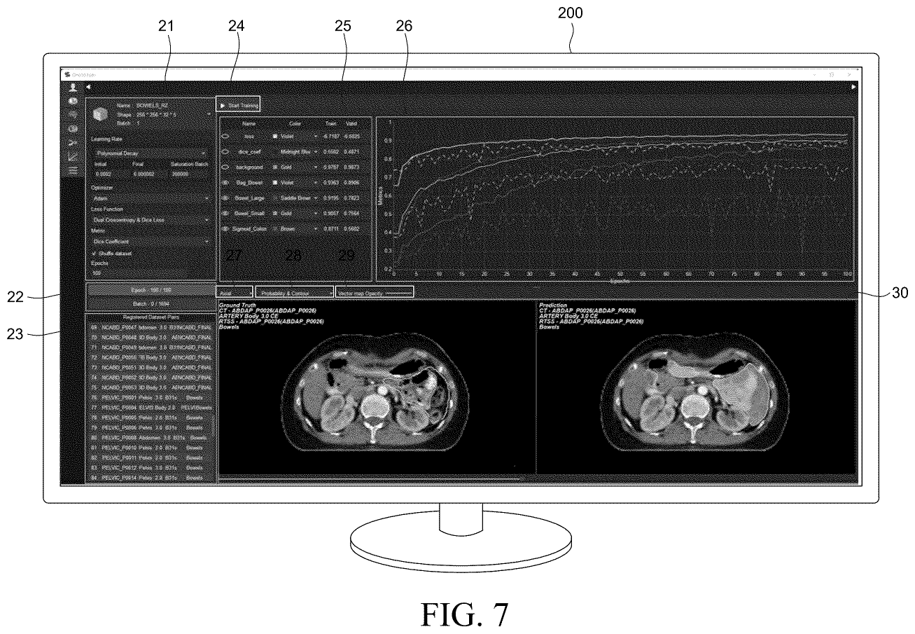

With regard to this, FIG. 7 is an exemplary view of a user interface screen which uses a prediction model for medical image segmentation according to an exemplary embodiment of the present disclosure.

Referring to FIG. 7, the processor 340 may train the first and second prediction models and provide a user interface screen which displays prediction results obtained by the first and second prediction models to the medical staff device 200. The user interface screen may include a region 21 which inputs and adjusts a learning parameter in the second prediction model. For example, batch size, iteration, and epoch parameters may be set by the user interface screen. The processor 340 may train the second prediction model based on a parameter input through the user interface screen and the user interface screen. The user interface may include a region 22 which displays a learning progress situation and a region 23 which displays a learning dataset used for the learning. Further, the user interface screen may include a graphic object 24 to train the second prediction model and a region 25 which displays a second prediction model list to perform the training as the graphic object 24 is selected. Further, in the region 25 which displays a second prediction model list to perform the training, a loss function and a scale to be used for the training may be displayed and a graphic object to select whether to visually display the prediction result may be included. Whenever a batch ends, a train value is updated. The processor 340 may update a valid value once the entire learning data is completely trained, that is, whenever each epoch ends. Further, the user interface screen may include a region 26 which displays a train value and a valid value on a graph for every second prediction model as the second prediction model is trained. For example, the train value may be represented with a solid line and the valid value may be represented with a dotted line. Further, the user interface screen may include a region 30 for displaying a prediction result of the target area using the second prediction model in the medical image. Here, the user interface screen may include a graphic object 27 for selecting which cross-section becomes a reference for displaying a region corresponding to the target area, a graphic object 28 for selecting which method is used to display a region corresponding to the target area, and a graphic object 29 for setting an opacity which displays a region corresponding to the target area in the medical image. Here, the user interface screen may emphasize only a region corresponding to the target area in the medical image and display a prediction result for each of the plurality of sub volume data, simultaneously.

The processor 340 may obtain any one reference plane to display the target area, among axial, coronal, and sagittal planes, through the user interface screen according to the choice of the medical staff. The processor 340 may render a region corresponding to the target area based on the reference plane to provide the region corresponding to the target area.

Until now, the medical image segmentation method performed by the processor 340 of the medical image segmentation apparatus 300 according to the exemplary embodiment of the present disclosure has been described. According to the present disclosure, the medical image segmentation apparatus 300 may not only input only a 2D image which configures the medical image to the first and second prediction models which predict a target area, but also provide front and rear image information of the 2D image which is sequentially captured through the volume data to improve a prediction accuracy of the model and expand the variety of the prediction result through two types of prediction models, more than predicting the target area solely using the prediction model.

Hereinafter, an overall flow of a training process and a prediction process of a prediction model which is performed by the medical image segmentation apparatus 300 will be described.

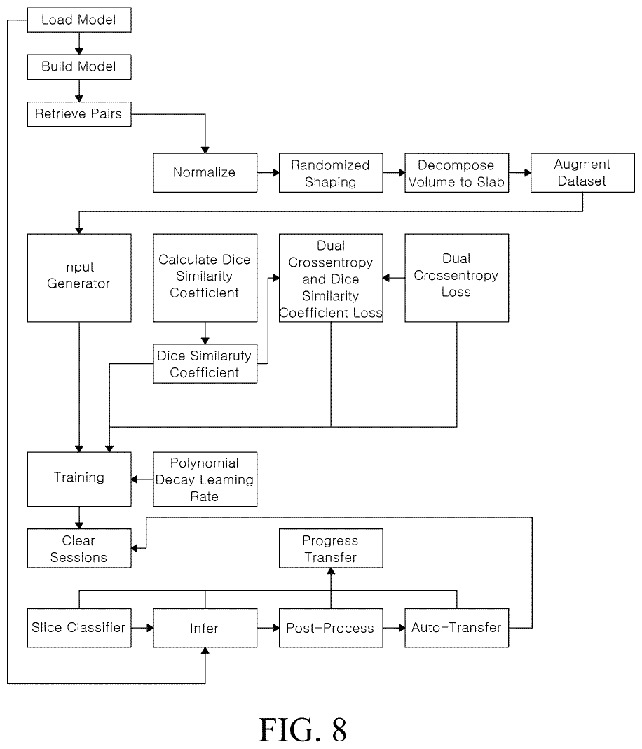

FIG. 8 is a schematic view for explaining a method for training a prediction model according to an exemplary embodiment of the present disclosure.

Referring to FIG. 8, the medical image segmentation apparatus 300 may preprocess a plurality of slices (learning dataset) used to train the second prediction model and then generate a plurality of sub volume data based thereon. The medical image segmentation apparatus 300 inputs the plurality of sub volume data to perform the learning and provide an operator to correct a difference between a learning result and an actual result.

As described above, the medical image segmentation apparatus 300 may train the second prediction model. Further, when a prediction request for a medical image is received in accordance with a request of a manager or a medical staff, the medical image segmentation apparatus 300 may load the second prediction model to segment a region corresponding to the target area in the medical image.

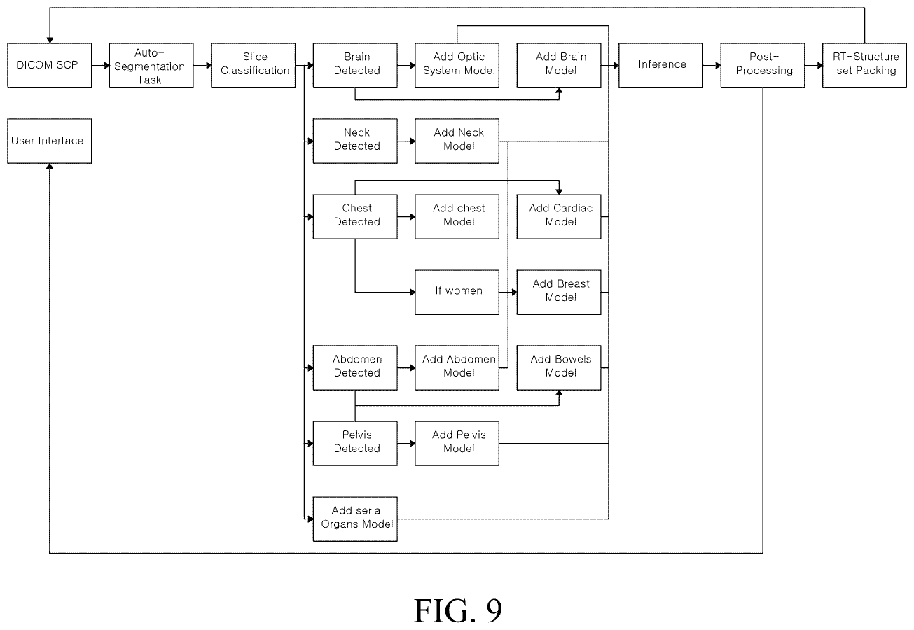

With regard to this, FIG. 9 is a schematic view for explaining an operation method of a prediction model which segments a medical image according to an exemplary embodiment of the present disclosure.

Referring to FIG. 9, the medical image segmentation apparatus 300 may acquire an enhanced and non-enhanced computed tomographic (CT) image which are generated according to the DICOM standard and confirm whether to perform auto-segmentation. For example, the medical staff device 200 may directly segment the target area from the medical image or request automatic segmentation, by a web or an application provided by the medical image segmentation apparatus 300.

If the automatic segmentation is requested, the medical image segmentation apparatus 300 may detect which target area is captured from a plurality of slices which configures the medical image using the classification model. That is, the medical image segmentation apparatus 300 may classify (or detect) a brain, a neck, a chest, an abdomen, or a pelvis, and other organs from the plurality of slices using the classification model and apply additional first and second prediction models according to a type of the target area or a type of a subject. For example, if the slice is classified as the brain, the medical image segmentation apparatus 300 may use first and second prediction models to additionally predict an optic area. As another example, if the slice is classified as the chest from a medical image of a female subject, the medical image segmentation apparatus 300 may use first and second prediction models to additionally predict a breast area. As still another example, if the slice is classified as the chest from a medical image of a subject having a cardiac disease, the medical image segmentation apparatus 300 may use a prediction model to additionally predict a heart area. As still another example, if the slice is classified as the abdomen and the pelvis, the medical image segmentation apparatus 300 may use first and second prediction models to additionally predict a bowel area.

As described above, the medical image segmentation apparatus 300 may classify and group the slices using the classification model. The medical image segmentation apparatus 300 may divide the grouped slices into a plurality of sub volume data and then predict whether each of the plurality of sub volume data corresponds to the target area using the first and second prediction models based thereon.

Although an exemplary embodiment of the present disclosure has been described in detail with reference to the accompanying drawings, the present disclosure is not limited thereto and may be embodied in many different forms without departing from the technical concept of the present disclosure. Therefore, the exemplary embodiments of the present disclosure are provided for illustrative purposes only but not intended to limit the technical concept of the present disclosure. The scope of the technical concept of the present disclosure is not limited thereto. Therefore, it should be understood that the above-described exemplary embodiments are illustrative in all aspects and do not limit the present disclosure. The protective scope of the present disclosure should be construed based on the following claims, and all the technical concepts in the equivalent scope thereof should be construed as falling within the scope of the present disclosure.

1. A medical image segmentation method performed by a processor of a medical image segmentation apparatus, comprising the steps of:
acquiring a medical image of a subject;
determining a first region corresponding to a target area by inputting the medical image to a first prediction model trained to predict a target area with a medical image as an input;
generating a plurality of sub volume data using the first region;
determining a second region corresponding to the target area in the first region from the plurality of sub volume data by inputting the plurality of sub volume data to the prediction model trained to predict the any one target area with a 3D medical image as an input; and
providing the second region corresponding to the target area in the medical image.

2. The medical image segmentation method according to claim 1, wherein the step of determining a second region is a step of determining a plurality of second regions corresponding to two or more different target areas according to a type of the target area corresponding to the first region.

3. The medical image segmentation method according to claim 1, wherein the sub volume data includes data regarding a moving direction of voxels configuring the sub volume data with respect to any one axis.

4. The medical image segmentation method according to claim 1, wherein the step of generating sub volume data is a step of generating the plurality of sub volume data by laminating a plurality of slices configuring the medical image at a predetermined height and segmenting the slices in a direction perpendicular to a plane of the slice.

5. The medical image segmentation method according to claim 1, further comprising the steps of:
prior to the generating of the plurality of sub volume data,
inputting the medical image to a classification model trained to classify a type of the target area with the medical image as an input;
determining the type of the target area included in each of a plurality of slices configuring the medical image; and
grouping the plurality of slices for every target area, according to a classification result.

6. The medical image segmentation method according to claim 1, wherein the step of providing a region corresponding to the target area is a step of providing a user interface screen including a segmentation model selection area to select the target area to be segmented in the medical image and a segmentation result display area to display the region corresponding to the target area.

7. The medical image segmentation method according to claim 6, wherein the step of providing a region corresponding to the target area further includes:
a step of combining volume data having a probability value corresponding to the target area equal to or higher than a predetermined value from the plurality of sub volume data to display the volume data on the user interface screen.

8.
The medical image segmentation method according to claim 6, wherein the step of providing a region corresponding to the target area further includes:
a step of displaying each of the plurality of sub volume data on the user interface screen with a different transparency according to a probability value corresponding to the target area, using the prediction model.

9. The medical image segmentation method according to claim 6, wherein in the step of generating the sub volume data, the plurality of sub volume data is generated with respect to an axis including any one plane, among axial, coronal, and sagittal planes, and
the step of providing a region corresponding to the target area further includes:
a step of acquiring any one reference plane to display the target area, among the axial, coronal, and sagittal planes, through the user interface screen; and
a step of rendering the region corresponding to the target area based on the reference plane.

10. The medical image segmentation method according to claim 1, further comprising the steps of:
prior to the step of acquiring a medical image,
acquiring learning data having a different slice thickness according to a type of the medical image;
generating a learning dataset by bootstrapping a different number of times for every thickness of the learning data; and
generating the prediction model configured to predict the target area based on the learning dataset.

11. A medical image segmentation apparatus, comprising:
a communication interface;
a memory; and
a processor operably connected to the communication interface and the memory,
wherein the processor is configured to acquire a medical image of a subject, determine a first region corresponding to a target area by inputting the medical image to a first prediction model trained to predict a target area with a medical image as an input, generate a plurality of sub volume data using the first region, determine a second region corresponding to the target area in the first region from the plurality of sub volume data by inputting the plurality of sub volume data to the prediction model trained to predict the target area with a 3D medical image as an input, and provide a second region corresponding to the target area in the medical image.

12. The medical image segmentation apparatus according to claim 11, wherein the processor is configured to determine a plurality of second regions corresponding to two or more different target areas depending on a type of the target area corresponding to the first region.

13. The medical image segmentation apparatus according to claim 11, wherein the sub volume data includes data regarding a moving direction of voxels configuring the sub volume data with respect to any one axis.

14. The medical image segmentation apparatus according to claim 11, wherein the processor is configured to generate the plurality of sub volume data by laminating a plurality of slices configuring the medical image at a predetermined height and segmenting the slices in a direction perpendicular to a plane of the slice.

15. The medical image segmentation apparatus according to claim 11, wherein prior to generating the plurality of sub volume data, the processor is configured to input the medical image to a classification model trained to classify a type of the target area with the medical image as an input, determine the type of the target area included in each of a plurality of slices configuring the medical image, and group the plurality of slices for every target area, according to a classification result.

16. The medical image segmentation apparatus according to claim 11, wherein the processor is configured to provide a user interface screen including a segmentation model selection area to select the target area to be segmented in the medical image and a segmentation result display area to display the region corresponding to the target area.

17. The medical image segmentation apparatus according to claim 16, wherein the processor is further configured to combine volume data having a probability value corresponding to the target area equal to or higher than a predetermined value, from each of the plurality of sub volume data, using the prediction model to display the volume data on the user interface screen.

18. The medical image segmentation apparatus according to claim 16, wherein the processor is further configured to display each of the plurality of sub volume data on the user interface screen with a different transparency according to a probability value corresponding to the target area, using the prediction model.

19. The medical image segmentation apparatus according to claim 16, wherein the processor is configured to generate the sub volume data with respect to an axis including any one plane, among axial, coronal, and sagittal planes, acquire any one reference plane to display the target area, among the axial, coronal, and sagittal planes, through the user interface screen to provide the region corresponding to the target area, and render the region corresponding to the target area based on the reference plane.

20. The medical image segmentation apparatus according to claim 11, wherein prior to acquiring the medical image, the processor is configured to acquire learning data having a different slice thickness according to a type of the medical image, generate a learning dataset by bootstrapping a different number of times for every thickness of the learning data, and generate the prediction model configured to predict the target area based on the learning dataset.
